(19) United States
(12) Patent Application Publication
(10) Pub. No.: US 2005/0255155 A1
(43) Pub. Date: Nov. 17, 2005

(54) Modified release cilostazol compositions

(75) Inventors: Nilendu Sen, Navi Mumbai (IN); Shrikant Bhonsle, Thane (IN); Kaliaperumal Arun Prasath, Navi Mumbai (IN); Anandi Krishnan, Navi Mumbai (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/125,584

(22) Filed: May 10, 2005

Related U.S. Application Data
(60) Provisional application No. 60/569,864, filed on May 11, 2004.

(51) Int. Cl. A61K 31/4709; A61K 9/48; A61K 9/20

(52) U.S. Cl. 424/451; 424/464; 514/312

(57) ABSTRACT

Pharmaceutical composition comprising a therapeutically effective amount of micronized particles of cilostazol or pharmaceutically acceptable salts or esters thereof, wherein at least about 50% of the micronized cilostazol particles have an effective average particle size of less than about 10 microns is provided.

### CROSS REFERENCE TO RELATED APPLICATIONS

This application claims the benefit under 35 U.S.C. §119 (e) to Provisional Application No. 60/569,864, filed May 11, 2004 and entitled “MODIFIED RELEASE CILOSTAZOL COMPOSITIONS,” the contents of which are incorporated by reference herein.

### BACKGROUND OF THE INVENTION

1. Technical Field

The present invention relates generally to modified release pharmaceutical compositions of cilostazol. More specifically, the present invention relates to modified release formulations containing micronized cilostazol particles.

2. Description of the Related Art

Cilostazol is known as 6-[4-(1-cyclohexyl-1H-tertazole-5-yl)butoxy]-3,4-dihydro-2 (1H)-quinolinone and has the following structure (Formula I): 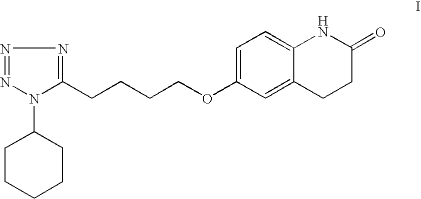
Cilostazol is a tetrazolylalkoxycarbostyril derivative and sold under the brand name Pletal®. Cilostazol has both antithrombic and vasodilating effects such that it is useful as an antithrombotic agent, a cerebral circulation improver, an anti-inflammatory agent, an anti-ulcer agent, a hypotensives agent, an antiasthmatic agent, and a phosphodiesterase inhibitor among other uses. See, e.g., The Merck Index, Thirteenth Edition, 2001, p. 395, monograph 2298; and Physician's Desk Reference, “Pletal,” 58^{th} Edition, p. 2500-2503 (2003).

The Biopharmaceutical Classification System (BCS) classifies cilostazol as a Class II drug which is characterized as having low solubility and high permeability. Oral absorption of cilostazol is rapid and complete. Kinetic profiles of cilostazol show that when in-vivo drug dissolution is complete, there is no constraint to absorption. Cilostazol has low solubility, and thus, the rate limiting step for oral bioavailability of cilostazol is the dissolution of the drug from the pharmaceutical dosage form.

Accordingly, there remains a need for improved pharmaceutical formulations of cilostazol which improve the dissolution of cilostazol, thereby increasing its bioavailability.

### SUMMARY OF THE INVENTION

In accordance with one embodiment of the present invention, a pharmaceutical composition is provided comprising a therapeutically effective amount of micronized particles of cilostazol or pharmaceutically acceptable salts or esters thereof, wherein at least about 50% of the micronized cilostazol particles have an effective average particle size of less than about 10 microns.

In accordance with a second embodiment of the present invention, a pharmaceutical composition is provided comprising a therapeutically effective amount of micronized particles of cilostazol or pharmaceutically acceptable salts or esters thereof, wherein at least about 70% of the micronized particles have an effective average particle size of less than about 10 microns.

In accordance with a third embodiment of the present invention, a pharmaceutical composition is provided comprising a therapeutically effective amount of cilostazol or pharmaceutically acceptable salts or esters thereof, wherein at least about 90% of the micronized particles have an effective average particle size of less than about 10 microns.

In accordance with a fourth embodiment of the present invention, a pharmaceutical composition is provided comprising a therapeutically effective amount of micronized particles of cilostazol or pharmaceutically acceptable salts or esters thereof, wherein at least about 95% of the micronized particles have an effective average particle size of less than about 10 microns.

In another embodiment of the present invention, a pharmaceutical composition is provided comprising a therapeutically effective amount of micronized particles of cilostazol or pharmaceutically acceptable salts or esters thereof, for oral administration, wherein the composition when administered to humans exhibits one or more of the following characteristics: (a) a maximum plasma concentration of about 1.1 to about 2.0 μg/ml; (b) an AUC 0-24 of about 13 to about 25 μg/ml; and (c) a Tₘₐₓ of about 2 to about 4 hours.

The pharmaceutical compositions containing the micronized particles of cilostazol herein advantageously may provides a modified release over an extended period of time as compared to conventional immediate release cilostazol unmicronized particles and compositions thereof.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention is directed to pharmaceutical compositions comprising micronized particles of cilostazol or pharmaceutically acceptable salts or esters thereof, wherein at least about 50% to about 95% of the micronized cilostazol particles have an effective average particle size of less than about 10 microns as measured by light-scattering methods or other conventional methods accepted in the art. The term “an effective average particle size of less than about 10 microns” as used herein shall be understood to mean that at least about 50% of the cilostazol particles have a weight average particle size of less than about 10 microns when measured by light scattering or other conventional techniques. Preferably, at least about 70% of the cilostazol particles have an average particle size of less than about 10 microns, more preferably at least about 90% of the cilostazol particles have an average particle size of less than about 10 microns, and even more preferably at least about 95% of the cilostazol particles have a weight average particle size of less than about 10 microns.

Cilostazol is well known and can be prepared according to known methods. See, e.g., U.S. Pat. No. 4,277,479. In accordance with the embodiment of the present invention, the particle sizes of the cilostazol prepared according to the present invention can be obtained by controlling the process of particle size reduction such as, for example, by air-jet milling or ceramic mill, to provide a finely divided powder, e.g. cilostazol having an effective average particle diameter of less than about 10 microns. In one embodiment, the process of particle size reduction can be controlled such that at least about 50% of the cilostazol particles will have an average particle diameter of less than about 10 microns. In another embodiment, the process of particle size reduction can be controlled such that at least about 70% of the cilostazol particles will have an average particle diameter of less than about 10 microns. In yet another embodiment, the process of particle size reduction can be controlled such that at least about 90% of the cilostazol particles will have an average particle diameter of less than about 10 microns. In still yet another embodiment, the process of particle size reduction can be controlled such that at least about 95% of the cilostazol particles will have an average particle diameter of less than about 10 microns.

The cilostazol thus obtained may then be formulated into a pharmaceutical composition or dosage form. Such compositions and dosage forms include, for example, compacted tablets, powder suspensions, capsules, and the like. The compositions of the present invention can be administered to humans and animals either orally, rectally, parenterally (intravenous, intramuscular, or subcutaneous), intracistemally, intravaginally, intraperitoneally, locally (powders, ointments or drops), or as a buccal or nasal spray. For example, the active ingredient of the invention, or salts or solvates thereof can be administered orally, buccally or sublingually in the form of tablets, capsules (including soft gel capsules), ovules, elixirs, solutions or suspensions, which may contain flavoring or coloring agents, for immediate-, delayed-, modified-, or controlled-release such as sustained-, dual-, or pulsatile delivery applications. The active ingredient of the invention may also be administered via fast dispersing or fast dissolving dosage forms or in the form of a high energy dispersion or as coated particles. Suitable pharmaceutical composition of the invention may be in coated or un-coated form as desired.

Actual dosage levels of the cilostazol or pharmaceutically acceptable salts or esters thereof in the compositions of the invention may be varied to obtain an amount of cilostazol that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. Generally, the amounts of micronized cilostazol present in the pharmaceutical composition of the present invention can range from about 30 to about 80% w/w and preferably from about 40 to about 70% w/w.

The following examples are provided to enable one skilled in the art to practice the invention and are merely illustrative of the invention. The examples should not be read as limiting the scope of the invention as defined in the claims.

### EXAMPLE 1

The cilostazol for use in this example was micronized by being passed through an air jet mill to where 90% of the cilostazol particles have a diameter of less than about 10 microns. The ingredients for this example are shown below in Table 1

TABLE 1

| Ingredients | Specifications | Qty/Tab (mg) |
|---|---|---|
| Cilostazol | Micronized | 100.0 |
| | (90% of particles | |
| | below 10 microns) | |
| Starch | Corn starch | 34.0 |
| Microcrystalline cellulose | Avicel PH 101 | 19.0 |
| Carmellose calcium | ECG-505 | 8.5 |
| Hydroxypropyl methyl | Methocel E3 LV | 6.8 |
| cellulose 2910 | | |
| Magnesium stearate | — | 1.7 |

A study was conducted between the formulation of Example 1 and Pletal.® The study was an open label, balanced, randomized two-treatment, two-period, two-sequence, crossover, comparative oral bioavailability study in 12 healthy adult male human subjects under fasting conditions. All of them completed the two-way crossover study. The results are set forth below in Table 2:

TABLE 2

| | | Least Square Means | | | | | |
|---|---|---|---|---|---|---|---|
| | | Reference | Test | | | 90% Confidence Intervals | |
| Parameter | Units | (3B94PAR3P) | (046/08-03/137A) | Ratio % | % IntraCV | Lower | Upper |
| Cmax | μg · hr/ml | 0.95 | 1.52 | 159.46 | 15.83 | 141.93 | 179.15 |
| AUC 0-t | μg · hr/ml | 14.98 | 18.93 | 126.43 | 17.58 | 111.11 | 143.85 |
| AUC 0-inf | μg · hr/ml | 15.55 | 19.37 | 124.57 | 17.70 | 109.38 | 141.86 |
| Tₘₐₓ | hr | 3.92 | 2.99 | 76.29 | — | 59.09 | 98.49 |
| t _{1/2} | hr | 6.69 | 5.33 | 79.69 | — | 68.62 | 92.55 |

The results show that the formulation of Example 1 had a higher Cmax T/R ratio of 159.46%, illustrating the superiority in the absorption profile (rate of absorption) of cilostazol in the formulation of Example 1 as compared to the Pletal® tablets. The T/R ratio of AUC 0-inf is 124.57%, which also shows that the total availability of cilostazol is greater in the formulation of Example 1 than Pletal®.

### EXAMPLE 2

90% of the particles of cilostazol used in this formulation was below 187 microns (unmicronized). The ingredients used in this example are shown below in Table 3:

TABLE 3

| Ingredients | Specifications | Qty/Tab (mg) |
|---|---|---|
| Cilostazol | Unmicronized | 100.0 |
| | (90% below 187 μ) | |
| Starch | Corn starch | 34.0 |
| Microcrystalline cellulose | Avicel PH 101 | 19.0 |
| Carmellose calcium | ECG-505 | 8.5 |
| Hydroxypropyl methyl | Methocel E3 LV | 6.8 |
| cellulose 2910 | | |
| Magnesium stearate | — | 1.7 |

A study was conducted between the formulation of Example 2 and Pletal®. The study was an open label, balanced, randomized two-treatment, two-period, two-sequence, crossover, comparative oral bioavailability study in 12 healthy adult male human subjects under fasting conditions. All of them completed the two-way crossover study. The results are set forth below in Table 4:

TABLE 4

| | | Least Square Means | | | | | |
|---|---|---|---|---|---|---|---|
| | | Reference | Test | | | 90% Confidence Intervals | |
| Parameter | Units | (2G70PAR2P) | (055/12-03/106) | Ratio % | % IntraCV | Lower | Upper |
| Cmax | μg · hr/ml | 0.75 | 0.43 | 56.64 | 23.80 | 46.93 | 68.35 |
| AUC 0-t | μg · hr/ml | 12.61 | 6.12 | 48.57 | 18.48 | 42.30 | 55.78 |
| AUC 0-inf | μg · hr/ml | 12.98 | 6.88 | 53.02 | 20.71 | 46.03 | 61.06 |
| Tₘₐₓ | hr | 5.20 | 8.88 | 170.64 | — | 126.65 | 229.91 |
| t _{1/2} | hr | 4.31 | 3.83 | 89.07 | — | 65.12 | 121.84 |

The results show the ratios of means for the cilostazol Ln-transformed parameters Cmax, AUC 0-t and AUC 0-inf were 56.64%, 48.57% and 53.02%. Comparing Examples 1 and 2 showed that the absorption of cilostazol is significantly dependent on the particle size of cilostazol.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A pharmaceutical composition comprising a therapeutically effective amount of micronized particles of cilostazol or pharmaceutically acceptable salts or esters thereof, wherein at least about 50% of the micronized cilostazol particles have an effective average particle size of less than about 10 microns.

2. The pharmaceutical composition of claim 1, wherein at least about 70% of the micronized cilostazol have an average particle size of less than about 10 microns.

3. The pharmaceutical composition of claim 1, wherein at least 90% of the micronized cilostazol have an average particle size of less than about 10 microns.

4. The pharmaceutical composition of claim 1, wherein at least 95% of the micronized cilostazol have an average particle size of less than about 10 microns.

5. The pharmaceutical composition of claim 1, further comprising one or more pharmaceutically acceptable excipients.

6. The pharmaceutical composition of claim 1, in the form of a powder suspension in a liquid.

7. The pharmaceutical composition of claim 1, in the form of a compacted tablet or a capsule.

8. The pharmaceutical composition of claim 5, in the form of a powder suspension in a liquid.

9. The pharmaceutical composition of claim 5, in the form of a compacted tablet or a capsule.

10. The pharmaceutical composition of claim 1, wherein the composition provides a modified release of the cilostazol particles.

11. The pharmaceutical composition of claim 2, wherein the composition provides a modified release of the cilostazol particles.

12. The pharmaceutical composition of claim 3, wherein the composition provides a modified release of the cilostazol particles.

13. The pharmaceutical composition of claim 4, wherein the composition provides a modified release of the cilostazol particles.

14. The pharmaceutical composition of claim 1, wherein the composition when administered to a human subject exhibits (a) a maximum plasma concentration of about 1.1 to about 2.0 μg/ml; (b) an AUC ₀₋₂₄ of about 13 to about 25 μg/ml; and (c) a Tₘₐₓ of about 2 to about 4 hours.

15. The pharmaceutical composition of claim 1, wherein the composition when administered to a human subject exhibits (a) a maximum plasma concentration up to about 1.52 μg.hr/ml; (b) an AUC_{0-inf} up to about 19.37 μg.hr/ml; (c) a Tₘₐₓ of about 2.99 hours.
